(19) United States
(12) Patent Application Publication
(10) Pub. No.: US 2010/0215786 A9
(43) Pub. Date: Aug. 26, 2010

(54) MORINDA CITRIFOLIA ENHANCED LIP TREATMENT

(76) Inventors: Claude Jarkae Jensen, Cedar Hills, UT (US); Heidi Robinson, Orem, UT (US)

Correspondence Address: KIRTON AND MCCONKIE, 60 EAST SOUTH TEMPLE,, SUITE 1800, SALT LAKE CITY, UT, 84111, US

(21) Appl. No.: 09/839,433

(22) Filed: Apr. 20, 2001

Related U.S. Application Data
(60) Provisional application No. 60/251,416, filed on Dec. 5, 2000.

(51) Int. Cl. A61K 35/78; A61K 7/025

(52) U.S. Cl. 424/765; 424/770; 424/64

(57) ABSTRACT

The present invention provides a lip treatment formulated with Morinda citrifolia juice or oil. The Morinda citrifolia lip treatment of the present invention serves to provide significant health and lip care advantages.

### BACKGROUND

1. Field of the Invention

The present invention relates generally to a protective formula for the skin and, more particularly, to a composition optimized for treating and rejuvenating the skin especially in application to the lips in the form of a lip balm or anti-chapping ointment.

2. Background

The skin on the lips is structurally different from skin elsewhere on a human's body. Lips have a thinner stratum corneum and a lesser amount of lipids than non-lip skin or typical skin. This allows water to easily pass through and dissipate from the lips. In fact, water loss through the lips is ten times greater than water loss through typical skin. This makes the lips more prone to dryness and, over time, to greater damage. This damage includes chapping, cracking, and sores that are not only painful, but unsightly to the individual.

Various compositions have been proposed to protect lips from drying and chapping as well as to promote healing should they become chapped. These compositions are effective up to a degree, but fail to promote prompt healing or provide effective protection against drying or chapping. For example, it has been found that lip treatments containing live yeast cell derivatives offer improved moisturizing effects as well as smoothing and healing the lips. These derivatives, however, must be processed and are expensive, which makes their use prohibitive.

Accordingly, consumers demand an effective treatment for the lips that moisturizes, heals, and soothes the vulnerable and delicate surface of the lips. Most lip products on the market consist of occlusive oils or waxes that limit the amount of moisture that escapes from the lips. These products do not address any underlying dryness, and merely prevent further chapping. Some products also contain moisturizing ingredients and healing ingredients. The success of these products varies widely.

### SUMMARY OF THE INVENTION

Protecting and beautifying the lips can be an important part of an overall daily skin care regime. To achieve soft and supple lips requires continuous care and attention. As many environmental conditions and elements exist which tend to dry and chap otherwise healthy lips, causing them to lose crucial moisture, protecting against these conditions and elements becomes important if one desires to maintain healthy lips. Recently, many health benefits of Morinda citrifolia have been discovered, including many benefits to the skin.

Therefore, it is an object of the preferred embodiments of the present invention to provide a lip treatment comprising Morinda citrifolia.

It is another object of the preferred embodiments of the present invention to provide a lip treatment comprising Morinda citrifolia for improved treatment of the lips.

It is still another object of the preferred embodiments of the present invention to provide a lip treatment comprising Morinda citrifolia for healing and preventing dry chapped lips.

It is a further object of the preferred embodiments of the present invention to provide a lip treatment comprising Morinda citrifolia for restoring vital nutrients and moisture to the lips.

It is still a further object of the preferred embodiments of the present invention to provide a lip treatment comprising Morinda citrifolia to prevent damage to and revitalize the lips.

The Morinda citrifolia enhanced lip treatment of the present invention provides numerous beneficial functions such as, revitalizing and smoothing the lips, restoring moisture to the lips, healing and protecting against dry chapped lips, protecting against environmental elements and conditions, and providing softer, more supple appearing lips. In addition to these beneficial functions, the Morinda citrifolia enhanced lip treatment of the present invention helps to reduce the visible signs of damaged or aged lips, while at the same time, meeting their specific treatment needs. Morinda citrifolia is high in antioxidants that help to fight free-radical damage caused by the sun and other elements. Morinda citrifolia is also rich in linoleic acid, which is an essential fatty acid with specific abilities for nourishing the health of the lips. Each of these, along with the other ingredients, aids in maintaining healthy lips.

Based on the foregoing objects, and in accordance with the invention as embodied and broadly described herein, a lip balm or lip treatment is disclosed that includes Morinda citrifolia, a carrier composition, and various other ingredients. The Morinda citrifolia may be present as Morinda citrifolia seed oil extract or fruit juice. In a preferred embodiment, the lip treatment comprises Morinda citrifolia seed oil. Morinda citrifolia fruit juice is processed from the puree, and the oil is processed from the seeds of the fruit.

The carrier composition can include, but is not limited to, petrolatum, ozokarite, octyl methoxycinnamate, cobitan oleate, benxophenone-3, bees wax, paraffin, canola oil, shea butter, propylparaben, tocopheryl acetate (vitamin E acetate), sweet almond oil, macadamia ternfolia seed oil, kukui nut oil, fragrance, butylated hydroxyanisole, propyl gallate, and citric acid. In a preferred embodiment, petrolatum combined with ozokerite are the main carriers. The lip treatment can be offered in the form of a lip balm, a lip ointment, a lipstick, or a lip gloss, among other suitable uses for lip application and treatment (hereinafter referred to collectively as “lip treatment”).

### DETAILED DESCRIPTION OF SPECIFIC EMBODIMENTS

It will be readily understood that the components of the present invention, as generally described herein, could be arranged and designed in a wide variety of different formulations. Thus, the following more detailed description of the embodiments of the compositions or formulations of the present invention are not intended to limit the scope of the invention, as claimed, but are merely representative of the presently preferred embodiments of the invention.

The present invention includes topical compositions that contain Morinda citrifolia as an ingredient in a suitable carrier for application to the lips.

### I. General Discussion of Morinda citrifolia

The Indian Mulberry or Noni plant, known scientifically as Morinda citrifolia L., is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Noni flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Noni plant was as a red and yellow dye source. Recently, there has been an interest in the nutritional and health benefits of the Noni plant, further discussed below.

The present invention utilizes the fruit juice and the oil extracted from the Morinda citrifolia plant. In a currently preferred process of producing Morinda citrifolia juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into finished juice product. The containers can be stored in refrigerated, frozen, or room temperature conditions. The Morinda citrifolia juice and puree are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp are further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The method for extracting and processing the oil is described in co-pending application Ser. No. 09/384,785, filed on Aug. 27, 1999, which is incorporated by reference herein. The Noni oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Noni plant is rich in natural ingredients. Those ingredients that have been discovered include: from the leaves: alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers: acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit: acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots: anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark: alizarin, chlororubin, glycosides (pentose, hexose), morindadiol, morindanigrine, morindine, morindone, resinous matter, rubiadin monomethyl ether, and soranjidiol; from the wood: anthragallol-2,3-dimethylether; from the tissue culture: damnacanthal, lucidin, lucidin-3-primeveroside, and morindone-6beta-primeveroside; from the plant: alizarin, alizarin-alpha-methyl ether, anthraquinones, asperuloside, hexanoic acid, morindadiol, morindone, morindogenin, octanoic acid, and ursolic acid.

Recently, many health benefits have been discovered stemming from the use of products containing Noni. The benefit of Noni is found in its ability to isolate and produce Xeronine, a relatively small alkaloid physiologically active within the body. Xeronine occurs in practically all healthy cells of plants, animals and microorganisms. Even though Noni has a negligible amount of free xeronine, it contains appreciable amounts of the precursor of xeronine, called Proxeronine. Further, Noni contains the inactive form of the enzyme Proxeronase which releases Xeronine from proxeronine. A paper entitled, “The Pharmacologically Active Ingredient of Noni” by R. M. Heinicke of the University of Hawaii, indicates that Noni is “the best raw material to use for the isolation of xeronine,” because of the building blocks of proxeronine and proxeronase. These building blocks aid in the isolation and production of Xeronine within the body.

Xeronine assists in enlarging the membrane pores of the cells. This enlargement allows for larger chains of peptides (amino acids or proteins) to be admitted into the cell.

Xeronine assists in enlarging the pores to allow better absorption of nutrients.

Each tissue has cells which contain proteins which have receptor sites for the absorption of xeronine. Certain of these proteins are the inert forms of enzymes which require absorbed Xeronine to become active. Thus Xeronine, by converting the body's procollagenase system into a specific protease, quickly and safely removes the dead tissue from skin.

Noni is said to provide a number of anecdotal effects in individuals having cancer, arthritis, headaches, indigestion, malignancies, broken bones, high blood pressure, diabetes, pain, infection, asthma, toothache, blemishes, immune system failure, and others.

### II. Morinda citrifolia Enhanced Lip Treatment

The present invention advances prior art lip treatments by providing a lip treatment formulated with Morinda citrifolia, or Noni, from the Indian Mulberry plant. The Morinda citrifolia is incorporated into various carriers suitable for application to the lips for application as a lip treatment. Morinda citrifolia seed oil is present in selected specific embodiments from about 0.1 to 20 percent by weight of the total weight of the composition. The carrier is present from about 10 to 80 percent by weight of the total composition. Morinda citrifolia fruit juice may also be present in an amount from about 0.1-50 percent by weight. Additional elements such as colorants, fragrances, and other ingredients, such as skin protectants, may also be present.

The present invention features a lip treatment comprising several key ingredients. These ingredients comprise Morinda citrifolia, or Tahitian Noni, and various other elements such as barrier repair technology elements, octyl methoxycinnamate, oxybenzone, octyl salicylate, and essential oils.

Morinda citrifolia, as mentioned above, is high in antioxidants that help fight free-radical damage to the skin caused by the sun and other elements. Morinda citrifolia is also especially rich in linoleic acid, an essential fatty acid with specific abilities to nourish the skin. This natural ingredient may also be combined with other natural ingredients containing antioxidants, such as Gingko biloba extract, grape seed extract, mushroom extract, and vitamins A, C, and E.

The composition according to the invention also comprises a cosmetically acceptable vehicle or carrier for the Morinda citrifolia in the composition, so as to facilitate its distribution when the composition or formulation is applied to the lips.

Carriers may include petrolatum, ozokerite, octyl methoxycinnamate, cobitan oleate, benxophenone-3, bees wax, paraffin, canola oil, shea butter, propylparaben, tocopheryl acetate (vitamin E acetate), sweet almond oil, macadamia ternfolia seed oil, kukui nut oil, fragrance, butylated hydroxyanisole, propyl gallate, and citric acid.

The cosmetically acceptable carrier will usually be present in an amount between about 10-80 percent by weight of the total composition. When multiple carriers are combined, these may be present in an amount between 1-40 percent by weight, and can, in the absence of other cosmetic adjuncts, form the balance of the composition.

Moisturizers that can be utilized in certain embodiments include lanolin, vegetable oil, castor oil, isopropyl palmitate, mineral oil, petrolatum, sweet almond oil, macadamia ternfolia seed oil, kukui nut oil, avocado oil, soy bean oil, caprylic/capric triglycerides, diisopropyl dimerate, methicone, or a combination thereof.

The lip treatment of the present invention also includes, in specific embodiments, a healing agent. Such healing agents can include, but are not limited to, octyl-methoxycinnamate, benzophenone-3, vitamin E, allantoin, candelilla wax, aloe vera, petrolatum, calamine, dimethicone, cocoa butter, shark liver oil, glycerin, zinc oxide, aluminum hydroxide, kaolin, zinc acetate, zinc carbonate, or a combination thereof.

The lip composition in accordance with the present invention, in specific embodiments, also may include, an occlusive wax. Specific waxes that may be included, but not limited to, are aloe wax, ceresine wax, ozokerite, lanolin wax, beeswax, paraffin, microcrystalline wax, animal wax, plant wax, synthetic wax, mineral wax, Japan wax, spermaceti, sunflower wax, orange wax, shellac wax, rice wax, jojoba wax, rose wax, jasmine wax, or a combination thereof. Lip compositions may include a colorant in specific embodiments or additional active ingredients. Ingredients may be selected from, but not limited to, aloe vera, elastin, collagen, vitamin E and derivatives thereof, vitamin A and derivatives thereof, liposomes, sodium hyaluronate, botanical extracts, phospholipids, silk powder, evening primrose oil, cholesteryl bio glutamate, hyaluronic acid, rose hips oil, lauroyl lysine, or a combination thereof.

The formulations of the present invention may also comprise sun screens. Sun screens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sun screen employed in the emulsions can vary depending upon the degree of protection desired from the sun's UV radiation.

Emollients are often incorporated into cosmetic compositions of the present invention. Levels of such emollients may range from 0.5% to 50%, preferably between 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as poly-pronylene glycol and polyethylene glycol. Butylene and propylene glycol are also especially preferred as penetration enhancers. Exemplary hydrocarbons which may serve as emollients are those having hydrocarbon chains anywhere from 12 to 30 carton atoms. Specific examples include mineral oil, petroleum jelly, squalene and isoparaffins.

Other adjunct minor components may also be incorporated into the cosmetic compositions. These ingredients may include flavors, coloring agents, opacifiers and fragrances. Amounts of these other adjunct minor components may range anvwhere from 0.001% up to 20% by weight of the composition.

The composition according to the invention is intended primarily as a product for topical application to human skin, specifically the lips. The composition is designed to provide protection, healing, conditioning, moisturizing and smoothing of the lips. The composition is also designed to soften and restore the lips to a healthy state, thus increasing their thickness, flexibility, elasticity, and overall appearance.

In use, a small quantity of the composition, for example from 1 to 100 ml, is applied to the lips from a suitable container or dispenser and, if necessary, it is then spread over and/or rubbed into the lips using an applicator or the fingers or some other suitable device.

The invention can be formulated as a lip balm, a lip ointment or gel, lipstick, lip gloss, or any other known lip treatment. The composition should be packaged in a suitable container to suit its viscosity and intended use by the consumer.

The following represent examples of lip treatment formulations comprising Morinda citrifolia extract therein to create an improved lip treatment formulation. These examples are merely illustrative and are not meant to be limiting in any way as one ordinarily skilled in the art will recognize the many ingredients that may be formulated with the Morinda citrifolia extract.

| | Percent by Weight |
|---|---|
| Active Ingredients | |
| Dimethicone | 1-5% |
| Octyl Methoxycinnamate | 5-10% |
| Octyl Salicylate | 1-5% |
| Oxybenzone | 1-5% |
| Inactive Ingredients | |
| Petrolatum | 40-70% |
| Ozokerite | 10-20% |
| Noni (Morinda citrifolia) Seed Oil | 1-10% |
| Cetyl Alcohol | 1-5% |
| Squalane | 1-5% |
| Butylated Hydroxytoluene | 1-5% |
| Menthol | 0-1% |
| Myristyl Lactate | 0-1% |
| Myristyl Myristate | 0-1% |
| Octyl Palmitate | 0-1% |
| Vitamin E | 0-1% |
| Flavor | 0-1% |

| | Percent by Weight |
|---|---|
| Active Ingredients | |
| Petrolatum | 40-70% |
| Octyl Methoxycinnamate | 5-10% |
| Oxybenzone | 1-5% |
| Inactive Ingredients | |
| Lanolin | 5-10% |
| Paraffin | 5-10% |
| Ozokerite | 5-10% |
| Noni (Morinda citrifolia) juice | 0.1-50% |
| Beeswax | 1-5% |
| Phenyl Trimethicone | 1-5% |
| Avocado Oil | 0-1% |
| Cetyl Alcohol | 0-1% |
| Chamomile Oil | 0-1% |
| Isopropyl Lanolate | 0-1% |
| Microcrystalline Wax | 0-1% |
| Propylparaben | 0-1% |
| Shea Butter | 0-1% |
| Zinc Oxide | 0-1% |
| Aloe Vera Extract | 0-1% |
| Methylparaben | 0-1% |
| Jojoba Oil | 0-1% |
| Flavor | 0-1% |

| | Percent by Weight |
|---|---|
| Active Ingredients | |
| Octyl Methoxycinnamate | 5-10% |
| Oxybenzone | 1-5% |
| White Petrolatum | 40-70% |
| Inactive Ingredients | |
| Isopropyl Myristate | 5-10% |
| Lanolin | 5-10% |
| Noni (Morinda citrifolia) Seed Oil | 1-10% |
| Waxes | 1-5% |
| Isopropyl Lanolate | 1-5% |
| Isocetyl Stearate | 1-5% |
| Fragrance | 0-1% |
| Cetyl Alcohol | 0-1% |
| Aloe Extract | 0-1% |
| Methylparaben | 0-1% |
| Propylparaben | 0-1% |
| Vitamin E Acetate | 0-1% |
| Vitamin E Linoleate | 0-1% |

| | Percent by Weight |
|---|---|
| Active Ingredients | |
| Benzophenone-3 | 1-5% |
| Octyl Methoxycinnamate | 5-10% |
| Octyl Salicylate | 1-5% |
| Petrolatum | 40-70% |
| Inactive Ingredients | |
| Ozokerite | 10-20% |
| Paraffin | 5-10% |
| Sorbitan Oleate | 1-5% |
| Noni (Morinda citrifolia) Seed Oil | 0.1-20% |
| Fragrance | 0-1% |
| Sodium Saccharinate | 0-1% |
| Sweet Almond Oil | 0-1% |
| Macadamia Ternifolia (Nut) Seed Oil | 0-1% |
| Kukui Nut Oil | 0-1% |
| Shea Butter | 0-1% |
| Camphor Gum | 0-1% |
| Menthol | 0-1% |
| Tocopheryl Acetate (Vitamine E Acetate) | 0-1% |
| Propylparaben | 0-1% |
| Propylparaben | 0-1% |

The Morinda citrifolia oil is incorporated with various carriers and other ingredients, as shown, suitable for application to the lips. Morinda citrifolia oil or extract may be present in selected specific embodiments between about 0.1-20 percent by weight of the total weight of the lip treatment or other composition.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed is:

1. A lip treatment comprising:
Morinda citrifolia seed oil present in an amount between about 0.1-20 percent by weight:

2. The lip treatment of claim 1, wherein said Morinda citrifolia seed oil is present in an amount between about 0.1 and 5 percent by weight.

3. The lip treatment of claim 1, wherein said Morinda citrifolia seed oil is present in an amount between about 5 and 10 percent by weight.

4. The lip treatment of claim 1, wherein said Morinda citrifolia seed oil is present in an amount between about 10 and 15 percent by weight.

5. The lip treatment of claim 1, wherein said Morinda citrifolia seed oil is present in an amount between about 15 and 20 percent by weight.

6. The lip treatment of claim 1, further comprising Morinda citrifolia fruit juice present in an amount between about 0.1-50 percent by weight.

7. The lip treatment of claim 1, further comprising dimethicone present in an amount between about 1-5 percent by weight.

8. The lip treatment of claim 1, further comprising octyl methoxycinnamate present in an amount between about 5-10 percent by weight.

9. The lip treatment of claim 1, further comprising octyl salicylate present in an amount between about 1-5 percent by weight.

10. The lip treatment of claim 1, further comprising oxybenzone present in an amount between about 1-5 percent by weight.

11. The lip treatment of claim 1, further comprising petrolatum present in an amount between about 20-80 percent by weight.

12. The lip treatment of claim 1, further comprising ozokerite present in an amount between about 5-20 percent by weight.

13. The lip treatment of claim 1, further comprising cetyl alcohol present in an amount between about 1-5 percent by weight.

14. The lip treatment of claim 1, further comprising squalane present in an amount between about 1-5 percent by weight.

15. The lip treatment of claim 1, further comprising butylated hydroxytoluene present in an amount between about 1-5 percent by weight.

16. The lip treatment of claim 1, further comprising lanolin present in an amount between about 5-10 percent by weight.

17. The lip treatment of claim 1, further comprising paraffin present in an amount between about 5-10 percent by weight.

18. The lip treatment of claim 1, further comprising beeswax present in an amount between about 1-5 percent by weight.

19. The lip treatment of claim 1, further comprising phenyl trimethicone present in an amount between about 1-5 percent by weight.

20. The lip treatment of claim 1, further comprising benzophenone-3 present in an amount between about 1-5 percent by weight.

21. The lip treatment of claim 1, further comprising sorbitan oleate present in an amount between about 1-5 percent by weight.

22. The lip treatment of claim 1, further comprising white petrolatum present in an amount between about 40-70 percent by weight.

23. The lip treatment of claim 1, further comprising isopropyl myristate present in an amount between about 5-10 percent by weight.

24. The lip treatment of claim 1, further comprising a wax derivative present in an amount between about 1-5 percent by weight.

25. The lip treatment of claim 1, further comprising isopropyl lanolate present in an amount between about 1-5 percent by weight.

26. The lip treatment of claim 1, further comprising isocetyl stearate present in an amount between about 1-5 percent by weight.

27. The lip treatment of claim 1, further comprising ingredients selected from the group consisting of Menthol, Myristyl Lactate, Myristyl Myristate, Octyl Palmitate, Vitamin E, Avocado Oil, Cetyl Alcohol, Chamomile Oil, Isopropyl Lanolate, Microcrystalline Wax, Propylparaben, Shea Butter, Zinc Oxide, Aloe Vera Extract, Methylparaben, Jojoba Oiland, Fragrance, Cetyl Alcohol, Aloe Extract, Methylparaben, Propylparaben, Vitamin B Acetate, Vitamin E Linoleate, Sodium Saccharinate, Sweet Almond Oil, Macadamia Ternifolia (Nut) Seed Oil, Kukui Nut Oil, Shea Butter, Camphor Gum, Menthol, Tocopheryl Acetate (Vitamine E Acetate), Propylparaben, and Flavor, each present in an amount between about 0-1 percent by weight.
